(19) United States
(12) Patent Application Publication
(10) Pub. No.: US 2019/0346406 A1
(43) Pub. Date: Nov. 14, 2019

(54) GAS DETECTION TECHNIQUE

(71) Applicant: RAMOT AT TEL-AVIV UNIVERSITY LTD., Tel Aviv (IL)

(72) Inventor: Alexander GERBER, Tel Aviv (IL)

(21) Appl. No.: 16/466,767

(22) PCT Filed: Dec. 5, 2017
(86) PCT No.: PCT/IL2017/051314

§ 371 Date: Jun. 5, 2019

Related U.S. Application Data
(60) Provisional application No. 62/430,600, filed on Dec. 6, 2016.
(60) Provisional application No. 62/564,457, filed on Sep. 28, 2017.

(51) Int. Cl. G01N 27/74; G01N 33/00; H01L 43/06; H01L 43/10

(52) U.S. Cl. CPC G01N 27/74; G01N 33/0006; H01L 43/10; H01L 43/06; G01N 33/005

(57) ABSTRACT

Technique for determining presence and/or concentration of a target gas by monitoring changes of Extraordinary Hall Effect (EHE) exhibited by an electrically conductive layer of a sensor element in presence of the target gas.

### FIELD OF THE INVENTION

The present invention relates to a gas detection technique, and particularly to solid state magnetic devices where the presence of a particular gas is detected by changes in magnetic or conductive properties of their sensing element.

### BACKGROUND OF THE INVENTION

Reliable detection of hazardous, harmful or toxic gases has become a major issue due to more stringent environmental and safety regulations worldwide. Solid state gas sensors present a high potential for applications where the use of conventional analytical systems such as gas chromatography or optical detection (e.g. by infrared radiation) is prohibitively expensive. The interaction between the analyte of interest in the surrounding gas and the solid state sensor material is transduced as a measurable electrical signal that most often is a change in the conductance, capacitance, or potential of the active element. According to the respective measurement type, these sensor devices are commonly classified as “potentiometric”, “amperometric”, “conductometric”, and so on.

Conductometric gas sensors based on semiconducting metal oxides are among the most common and widely used groups of gas sensors. They attracted many users due to low cost and flexibility associated to their production; simplicity of use and large number of detectable gases and possible application fields. Operation of these sensors is based on a change of electric conductivity of some metal oxide semiconductor materials such as SnO₂, NiO, and Cr₂O₃ when exposed to an atmosphere containing specific gases.

Important disadvantages of the solid state sensors are their sensitivity to water vapor and the lack of selectivity. Metal oxide-based gas sensors are normally sensitive to more than one chemical species in air and usually show cross-sensitivities. This non-specificity of the response to chemical species whose presence, identity and concentration in air have to be determined is by now considered an intrinsic property of metal oxide-based gas sensors. This disadvantage represents a real problem when different reactive gases are present simultaneously in the same atmosphere so that interference effects between them may occur. In addition, the adsorption process that is responsible for the sensor signal is strongly influenced by the presence of the pre-adsorbed species (like ionosorbed oxygen, hydroxyl groups, carbonates, etc.).

When the only parameter measured by the sensor is the change of resistance upon exposure to the target gas, one can only record the overall electrical effect of quite complex surface reactions. In other words, by only measuring the resistance change one does not have the needed discrimination for the correlation between specific surface species and their electrical effect. In principle, the needed discrimination can be provided by the results obtained by applying additional spectroscopic techniques. However, most of the standard spectroscopic investigations are to be performed in conditions far away from the ones normally encountered in real sensors applications, namely: in ultra-high vacuum; at low temperatures; required preconditioning of the samples at high temperatures, quenching and exposure to high concentrations of reactive gases; conducted on simplified systems, etc.

The concept of magnetic gas detection has been promoted by a number of researchers [1], [2] who discovered that magnetic properties of several materials are modified when exposed to certain gases, for example to hydrogen. Interaction of hydrogen with ferromagnetic structures containing Pd was shown to change their structural, electronic, optical, and magnetic properties. Modifications in magnetization, coercive field, squareness of hysteresis loop, optical Kerr signal and magnetic anisotropy were found in Co/Pd multilayers, Pd/Co/Pd tri-layers, Pd/Fe, Pd/Co and Pd/Ni bilayers and in Pd-rich CoPd alloy films. Additional materials like Fe/Nb, Fe/V superlattices and SnFeO₂ ferrites also demonstrated systematic changes in magnetic properties and exchange coupling when loaded with hydrogen. All effects mentioned above were detected by using standard laboratory magnetometric techniques and equipment like neutron reflectivity, X-ray resonant magnetic scattering (XRMS), superconducting quantum interference devices (SQUID), vibrating magnetometers or optical Kerr effect measurements. Adaptation of the mentioned techniques to field conditions present a formidable challenge.

In view of the above, although the concept of magnetic gas sensing (using the magnetic property of a material as a gas sensing parameter) has been formulated, its realization in practical/field sensor devices was not implemented so far.

At the same time, in studies of magnetic properties of ultra-thin magnetic films and nano-structures, a so-called Hall effect, and more specifically—Extraordinary Hall effect (EHE) or Anomalous Hall effect (AHE) was pointed out as a highly sensitive tool. Very high sensitivity to external magnetic field was demonstrated for materials with artificially enhanced EHE coefficient R_{EHE}—but only for applications as sensors of magnetic field [3].
Object and Summary of the Invention

It is an object of the invention to provide a technique which would allow developing a simple and effective magnetic gas sensor having sensitivity and selectivity which are required for practical, field measurements. The technique also comprises a device including such a sensor, a method for detecting gases by such a sensor and/or device, and a software product suitable for use in the proposed device.

According to a first aspect of the invention, there is provided a method for determining presence and/or concentration of a target gas by monitoring changes of Extraordinary Hall Effect (EHE) exhibited by an electrically conductive layer of a sensor element in presence of the target gas.

The electrically conductive layer (which is also magnetic, e.g. may be magnetized) should be capable of exhibiting Extraordinary Hall Effect (EHE), i.e. if electric current passes through a sample/layer of such a magnetized material, the sample is expected to exhibit EHE in a direction perpendicular to the current's direction.

More specifically, the Inventor has proposed, for determining presence and concentration of a target gas, to monitor (without and supposedly with said gas) the EHE exhibited by the electrically conductive and magnetized sample/layer in a direction perpendicular to direction of an electric current passing through said layer, and to use changes of EHE (if any) for said detection.

It has been proven by the Inventor, that changes in the EHE may serve as an effective independent or complementary criterion for detecting target gases.

In the frame of the present description, the term “electrically conductive layer” should be understood as an electrically conductive sample/unit of any form, which may constitute a plate, a strip, a film, etc. The terms “layer”, “unit”, “sample” and “film” will be used intermittently in the description. It should also be kept in mind that an electrically conductive unit may be a multilayered structure.

Yet more specifically, the method may comprise:
providing the sensor element comprising the conductive layer capable of exhibiting Extraordinary Hall Effect (EHE),
exposing the sensor element to an atmosphere comprising the target gas (may be performed in advance and/or while the conductive layer is connected to an electrical power source)
magnetizing the sensor element (may be performed in advance and/or while the conductive layer is connected to an electrical power source)
connecting said conductive layer to an electrical power source (for providing passage of electric current through said conductive layer),
monitoring EHE to detect supposed changes in the EHE of said conductive layer upon said exposure,
in case the changes in the EHE are detected, defining presence and concentration of said target gas by assessing said changes.

The method may comprise obtaining or preliminarily building a database comprising records on changes of EHE of the conductive layer in the presence of said target gas at one or more reference concentrations thereof. The database can be used for calibration of the sensor element.

The monitoring of EHE (and thus of EHE changes) may be performed by measuring a Hall effect signal (“Hall signal”). More specifically, the method may be performed as follows:
magnetizing the sensor element using external magnetic field,
providing passage of electric current along said conductive layer (say, by injecting electric current along the sensor element),
monitoring EHE of the sensor element by measuring a first electric signal being a Hall effect signal (appearing in a direction perpendicular to direction of the injected electric current), and
defining the presence and concentration of the target gas by processing the measured first electric signal.

In the frame of the present description and claims, the first signal should be understood as a Hall effect signal exhibiting, inter alia, the mentioned Extraordinary Hall effect (EHE) which appears owing to action of the magnetic field on the sensor element (which, as we agreed, is capable of demonstrating EHE). The Hall effect signal can be measured, for example, by measuring Hall voltage which occurs in a direction perpendicular to the direction of electric current injected in the sensor element. The Hall voltage indicates the so-called Hall effect resistance of the sensor element in a direction perpendicular to the electric current direction. It should also be kept in mind that Hall current may be measured instead of Hall voltage, for example via short-circuited electrical contacts which otherwise could be used for measuring Hall voltage. In the description and claims, information on the measured Hall effect signal may be further referred to as Hall effect data.

The method may comprise exposing the sensor element to external magnetic field before or at the time of said EHE monitoring. The same timing applies to exposing the sensor element to the target gas/gases. As noted before, the EHE monitoring may comprise measuring the first signal (a Hall effect signal).

In the method, magnetic field may be applied perpendicularly to the plane of the sensor element.

In the proposed novel technique (method, sensor, device and protocol/software product for device operation), the monitoring of the magnetic properties of the sensor is performed by measuring the so-called Hall signal, in particular—Hall voltage which indicates the extraordinary Hall effect (EHE).

The measured Hall voltage always comprises a contribution of EHE and the ordinary Hall effect. (For example, one can get a contribution of the longitudinal resistance if the Hall contacts are not strictly opposite to each other).

In the terms of the proposed method, said monitoring of EHE may be performed by measuring Hall voltage V_{H} across the sensor element in a direction perpendicular to the electric current direction.

The essence of the effect is the following: electric current flowing along magnetic film (see FIG. 1) generates a Hall voltage in a direction perpendicular to the current direction given by:

$${V}_{H} = \frac{I}{t} {ρ}_{H} = \frac{I}{t} ( {R}_{0} B + {R}_{EHE} {μ}_{0} M ) \qquad ( 1 )$$

where I is current, t thickness of the film, ρ_{H} is the Hall resistivity, B, and M are components of the magnetic induction and magnetization normal to the plane of the conductive layer (film). R₀ is the ordinary Hall coefficient related to the Lorentz force acting on moving charge carriers. R_{EHE}, the extraordinary Hall coefficient, is associated with a break of the right-left symmetry at spin-orbit scattering in magnetic materials. In cases of our interest the EHE contribution can exceed significantly the ordinary Hall effect term in the low field range (actually the range of values below 1 Tesla, i.e. a practical range feasible for gas sensors), and the total Hall voltage V_{H} can be approximated as:

V_{H}=V_{EHE}=R_{H}^{I}=μ₀R_{EHE}MI/t (2)

Thus, the Hall voltage is directly proportional to magnetization. Though such effect was known and used for studies of magnetic properties of ultra-thin magnetic films and nano-structures, it was never proposed as a tool for gas detection. It should also be mentioned, that the proposed gas sensor may actually utilize magnetic layers of any size.

In this invention, the Inventor proposes to use the Hall effect (more specifically, the extraordinary Hall effect) as a tool to monitor changes in magnetic properties caused by exposure of the sensor material to a specific gaseous element. Changes affecting either magnetization, or the EHE coefficient can be detected by variations in the measured Hall voltage V_{H}. The measurement technique is technically similar to measurement of resistance in the existing conductometric sensors, but has two major modifications: 1) Hall voltage V_{H} is measured in a direction perpendicular to the electric current flow (not parallel to the current flow as in the resistance measurement) and 2) the measurement of V_{H} is done in magnetic field generated by e.g. an attached permanent magnet or by an electromagnet (in certain cases the measurement can also be done at zero magnetic field, for example after pre-magnetizing the sensor element). Thus, in the proposed technique it is essential to monitor changes in the magnetic properties of the sensor element by measuring a Hall signal (say, EHE signal in the form of V_{H}).

For increasing sensitivity and selectivity of the sensing element, the method may further comprise: monitoring changes of conductivity properties of the sensor element (for example, by measuring a second electric signal indicating resistance along the sensor element), and defining the presence and concentration of the target gas by processing, together, the changes of EHE and the changes of conductivity properties (for example, by processing the measured signals: a first, Hall effect signal and a second electric signal indicating resistance along the sensor element).

The second electric signal may be, for example, voltage measured along the sensor element to obtain the so-called longitudinal resistance of the sensor element, wherein the value of the current (being DC or AC current) is known.

More specifically, the method may comprise

injecting the electric current crossing the sensor element in a longitudinal direction;

monitoring the conductivity properties by measuring electric voltage V_{XX} along the sensor element in the longitudinal direction;

monitoring EHE by measuring Hall voltage V_{H} across the sensor element in a direction perpendicular to the longitudinal direction;

judging about the presence and concentration of said target gas by processing the measured values of electric voltage V_{XX} and Hall voltage V_{H}.

The EHE signal can be measured quite simultaneously with resistance in the same setup. Thus, in the disclosed technique, two properties can be monitored simultaneously and measured actually simultaneously: resistivity and Hall effect, the latter proportional to magnetization of the sensing element.

In other words, the proposed technique of magnetic gas detection by the Extraordinary Hall effect (EHE) is compatible with the existing conductometric gas detection technologies and allows simultaneous measurement of two independent parameters: resistivity and magnetization affected by the target gas.

Any of said measurements (of the Hall signal; of resistivity and of the Hall signal) can be performed under some predetermined magnetic field.

Alternatively or in addition, any of said measurements may be performed under a sequence of predetermined magnetic fields.

In one version of the method, for detecting presence of hydrogen it may include using the electrically conductive layer comprising a material selected from CoPd, FePd, NiPd or any combination thereof, —for example in the form of film(s). Moreover, the Inventor has shown that thin films may be especially useful for detecting low concentrations of hydrogen, for example from 0% to of about 4%.

In the method described above, for increasing sensitivity, the electrically conductive layer (unit) may be actually multilayered and constitute a so-called stack (i.e., be provided to comprise multiple films as sub-layers made of the same material or composition of materials, for example, with protecting layers between the films); said stack having a common input contact and a common output contact and exhibiting EHE changeable in presence of the target gas. In such a case the step of monitoring will be performed for the stack to determine presence and concentration of the target gas.

In an alternative version of the method, the method may comprise providing at least one additional electrically conductive unit made of a different material or composition of materials than the basic electrically conductive layer, said additional unit being capable of exhibiting Extraordinary Hall Effect (EHE) changeable in presence of said target gas or an additional target gas; such a version of the method comprises performing the monitoring step for the basic electrically conductive layer and for the additional electrically conductive unit separately.

If the at least one additional conductive unit is sensitive to the same said target gas, said alternative version of the method may be used for determining presence and concentration of the target gas with more selectivity.

If said at least one additional conductive unit is sensitive to an additional target gas (i.e., exhibits EHE changeable in presence of the additional target gas), said alternative version of the method may be used for determining presence and concentration both of said target gas and of said additional target gas.

It is understood, that the above-described different versions of the method will comprise respectively suitable versions of processing the monitoring results.

According to a second aspect of the invention, there is provided a gas sensor element comprising an electrically conductive layer capable of exhibiting Extraordinary Hall Effect (EHE) which is expected to be changeable in the presence of a target gas.

The conductive layer of the sensor element may be capable of exhibiting the EHE upon being magnetized and exposed to electric current through said conductive layer. Sensitivity of the sensor element may be increased if said conductive layer of the sensor element is designed/selected to have also its conductivity properties changeable by presence of the target gas.

In other words, both resistivity and the extraordinary Hall effect (EHE) of the sensor element may respond to the presence of the target gas.

The electrically conducting layer of the sensor element may comprise a material which can be magnetized, for example one or more (i.e., a combination) of the following materials: ferromagnetic materials, like ferromagnetic metals or a ferromagnetic semiconductors, paramagnetic materials; may constitute bilayers or multilayers composed of magnetic materials and/or non-magnetic metals, alloys or mixtures of magnetic and/or non-magnetic materials.

Examples of the materials, which can be used in a sensor element for detection of presence and/or concentration of hydrogen, will be given further below.

The electrically conductive layer may be a film, for example a thin film. Thin films have been shown to be more effective for detecting low concentration of gases, at least for a number of examples. It has been shown by the Inventor, that the film thickness should be such as to ensure that the film is electrically conductive. In practice, the film having thickness from of about 1 nm demonstrates electrical conductivity.

The sensor element may have a so-called Hall bar geometry with at least five contact terminals. For example, there may be two current contacts, two longitudinal voltage measurement contacts and at least one additional contact for transverse Hall effect measurement.

Alternatively, the sensor element may have an arbitrary planar shape and four contacts allowing the Van der Pauw measurement protocol.

Further, the gas sensor element may comprise two or more electrically conductive units, each of them exhibiting EHE being changeable in response to a specific target gas.

In one embodiment, said two or more electrically conductive units may be all responsive to one (said) target gas. It should be mentioned, however, that such conductive units may be made of the same material/combination of materials, but may be not.

The mentioned two or more conductive units, if all are responsive to the same said target gas and all are made of the same material/composition of materials, may be arranged in the form of a stack of conductive layers divided by protective layers and having a common input contact and a common output contact for injecting electric current through the multiple conductive layers. In the stack, the Hall effect signal can be measured as a superposition of signals of all such layers.

Such a stack of layers (say, ferromagnetic films) facilitates creation of magnetic hysteresis in the gas sensor element under external magnetic field. The described multiple conductive layers of the stack may also increase sensitivity and selectivity of the sensor.

However, in another embodiment of the gas sensor element, the two or more conductive units may be made of different materials/compositions so as to target one gas common to them. In other words, such two or more different units are sensitive to one and the same target gas.

In such an embodiment of the gas sensor element, each of the units should be positioned and tested (monitored) separately. Preferably, such conductive units should be located in the sensor so as to avoid overlapping of one unit by another and thus to allow maximal exposure of each unit to the target gas. Such a solution may improve selectivity of the gas sensor element, due to detection of the target gas by alternative conductive units of the gas sensor element.

Still further, if the task is to target different gases, a set (or an array) of gas sensor elements may be provided. The set (system) may comprise two or more of the above-described sensor elements for respectively detecting presence of two or more different target gases, wherein each of said two or more sensor elements being designed according to any of the versions described above. In such a set/array intended for detection of different gases, each conductive unit (or stack of layers) should be tested separately. Also in that embodiment, conductive units or stacks should not overlap one another.

For example, the gas sensor element may be designed for detecting presence of hydrogen, to this end it may include the electrically conductive layer comprising a CoPd, a FePd and/or a NiPd film(s), for example thin films. Such a gas sensor element may be useful for detecting low concentrations of hydrogen from 0% to about 4%, be especially useful for detecting concentrations between 0% and of about 1%, and be most sensitive to concentrations below of about 0.5%.

The Inventor has found that the CoPd film of interest may be in the form of a Co/Pd bi-layer, a Co/Pd multilayer, a Co-Pd alloy or any combinations thereof.

The Inventor has further found that the CoPd film may have the Co volume concentration in the range of about 3% to 45%, for example when the film is in the form of the alloy or the multilayer.

According to a further aspect of the invention, there is provided a device for defining presence and/or concentration of a target gas.

It is a device comprising a gas sensor element having an electrically conductive layer capable of exhibiting Extraordinary Hall Effect (EHE) changeable in the presence of a target gas; the device being adapted to monitor changes of the EHE for determining presence and concentration of the target gas.

The device may include:
the sensor element (i.e., the sensor element as described above),
a magnetic field generator for magnetizing the sensor element,
contacts for connecting said conductive layer to an electric power source (for example, so as to inject electric current along the conductive layer),
a sensing (measuring, monitoring) unit comprising at least a first circuit for monitoring EHE by measuring a first electric signal being a Hall effect signal; changes of the first electric signal being indicative of presence and concentration of the target gas.

The device may further comprise a processing (analytic) unit configured to determine the presence and concentration of the target gas based on changes of the first electric signal.

The device may also include an electric power source (for example a standard battery).

The device may further comprise a display unit connected to the monitoring unit or to the processing unit.

The sensing unit of the device may further comprise a second circuit for monitoring conductivity of the conductive layer by measuring a second electric signal indicative of resistance along the conductive layer, and the processing unit configured to define the presence and concentration of the target gas based on changes of the first electric signal and changes of the second electric signal.

In one specific embodiment, the proposed device may comprise:
the first circuit for monitoring Hall voltage V_{H} in a direction perpendicular to the current direction (say, across the sensor element), and a second circuit for monitoring electric voltage V_{XX} in the direction of electric current(say, along the sensor element);
the processing unit for processing the monitored values of electric voltage V_{XX} and Hall voltage V_{H} and for judging about the presence and concentration of said at least one target gas, based on the processing results.

In the device ready to operation, the conductive layer (e.g. a ferromagnetic film) may be electrically connected to the electric power source so that an electrical current traverses the film and generates respective two voltage signals: one along the electric current path and the other transverse to the current path. The voltage signal V_{XX} along the current path indicates the longitudinal electrical resistance of the sensor; and the voltage signal transverse to the current path is the Hall voltage V_{H}, which indicates the Hall effect signal (being the Hall Effect data). As mentioned above, V_{H} measured in the direction substantially perpendicular to direction of injected current will be indicative either of the Extraordinary Hall effect (EHE), also known as the Anomalous Hall effect (AHE), or of a superposition of the Ordinary and the Extraordinary Hall effects. The sensing circuit may be configured to read out the resistance and the Hall effect data separately (but preferably simultaneously) by measuring the first and second voltages V_{XX} and V_{H}. The processing/analytic unit may be configured to define the presence and concentration of the target gas based on the measured resistance and Hall effect data.

In some embodiments, the device may include a current source that is configured to generate the electrical current and provide the electrical current to the film. In one embodiment, the device includes a conductor that connects the films, and the current source is configured to apply the electrical current so as to traverse the films and the conductor.

In one embodiment, the magnetic field generator is configured to produce a constant magnetic field applied to the conductive layer (ferromagnetic film).

In a different embodiment, the magnetic field generator may be configured to produce the continuously variable magnetic field or a sequence of magnetic field pulses, so as to alternate the magnetic field in polarity and in magnitude along the sequence.

In another embodiment, the processing unit may be configured to apply a reverse magnetic field reciprocity (RMFR) theorem to the measurements of V_{H} collected from the layer(s)/film(s), so as to separate from the really measured signal of V_{H} (as from a vector), the measure component(s) of V_{XX} indicating the longitudinal resistance, and component(s) of the Hall voltage V_{H}.

The device may be equipped with a heater, a thermometer and/or a temperature control circuit so as to vary and maintain the desired temperature of the sensor unit. (It should be noted that some conductive layers absorb gases beginning from a specific temperature.)

The device may be designed to accommodate two or more gas sensor elements sensitive to respective target gases; the measuring unit and the processing unit of the device may be accordingly designed to ensure monitoring and processing of at least changes in EHE respectively exhibited by said two or more sensor elements.

It should be noted that the above-mentioned respective target gases may be different target gases.

The gas sensor element of the device may comprise the electrically conductive layer being a CoPd, a FePd and/or a NiPd film; such a device will be adapted for detecting hydrogen, even low concentration hydrogen (for example between 0 and 1%).

According to yet a further aspect of the invention, there is also provided a software product comprising computer implementable instructions and/or data for carrying out the above-described method, stored on an appropriate non-transitory computer readable storage medium so that the software is capable of enabling operations of said method when used in a computerized system (being for example the sensor device).

### BRIEF DESCRIPTION OF THE DRAWINGS

The invention will further be explained and illustrated with the aid of the following non-limiting drawings, in which:

FIG. 1 is a schematic illustration of an exemplary setup according to the invention, actually being one embodiment of the proposed gas sensor utilizing Extraordinary Hall effect (EHE).

FIG. 2 is a schematic illustration of the Extraordinary Hall effect (EHE) measured as a function of magnetic field at different concentrations of a target gas, when the sensor element (conductive layer or film) exhibits no hysteresis effect in magnetic field.

FIG. 3 is a schematic illustration of the Extraordinary Hall effect (EHE) measured as a function of magnetic field at different concentrations of a target gas, while the sensor film exhibits the out-of-plane magnetic anisotropy and a hysteresis effect in the magnetic field.

FIG. 4 is a zoom of the data which could be found around a saturated portion of a hysteresis loop in positive magnetic fields, with points D, E and F indicating three possible exemplary working points of the proposed sensor device.

FIG. 5 illustrates field dependence of the Hall resistivity of CoₓPd₁₋ₓ films with x=0.08, 0.15, 0.2 and 0.25 (atomic concentrations) and respective thickness 15 nm, 18 nm, 15 nm and 14 nm measured in ambient air at room temperature.

FIGS. 6, 7, 8 present examples of Hall effect resistance as a function of magnetic field of CoₓPd₁₋ₓ films measured in air (open/bright circles) and in hydrogen/nitrogen H₂/N₂ mixture with 4% of hydrogen (solid circles). The examples are as follows:

FIG. 6 presents said function for a Co_{0.08}Pd_{0.92} film;

FIG. 7 presents the mentioned function for a Co_{0.2}Pd0.85 film;

FIG. 8 presents the mentioned function for a Co_{0.2}Pd_{0.8} film.

FIG. 9 presents EHE resistance hysteresis loops measured in 5 nm thick Co_{0.17}Pd_{0.83} film in H₂/N₂ atmosphere with different H₂ concentrations (y=0%, 0.125%, 0.25%, 0.5%, 1%, 2% and 4%).

FIG. 10 presents dependence of the coercive field Hc on Hydrogen concentration “y”.

FIG. 11 shows Hydrogen concentration dependence of the normalized EHE change ΔR_{H,norm} under bias fields 0 mT, 1.5 mT and 4 mT within the hysteresis loop.

FIG. 12 shows Resistance response to hydrogen, measured simultaneously with the EHE response to hydrogen for an exemplary proposed gas sensor element.

FIG. 13 presents a general schematic illustration of one embodiment of the devise comprising the proposed gas sensor and a magnetic field generator.

FIG. 14 is a schematic block diagram of another embodiment of the proposed device for gas detection.

### DETAILED DESCRIPTION OF SPECIFIC EMBODIMENTS

The invention will be described with reference to an exemplary sensor body which, according to one embodiment of the present invention, comprises a ferromagnetic layer/film exhibiting extraordinary Hall resistivity sensitive to the presence and quantity of the selected gas in atmosphere. Sensitivity of the EHE to the presence of the target gas can be the result of changes in the absolute saturated magnetization of the sensor material, in magnetic anisotropy, in magnetic field susceptibility or in general field dependence of magnetization. Alternatively, the EHE response to the target gas can be the result of variation in the extraordinary Hall effect coefficient, or to combination of changes in magnetic properties and the extraordinary Hall effect coefficient.

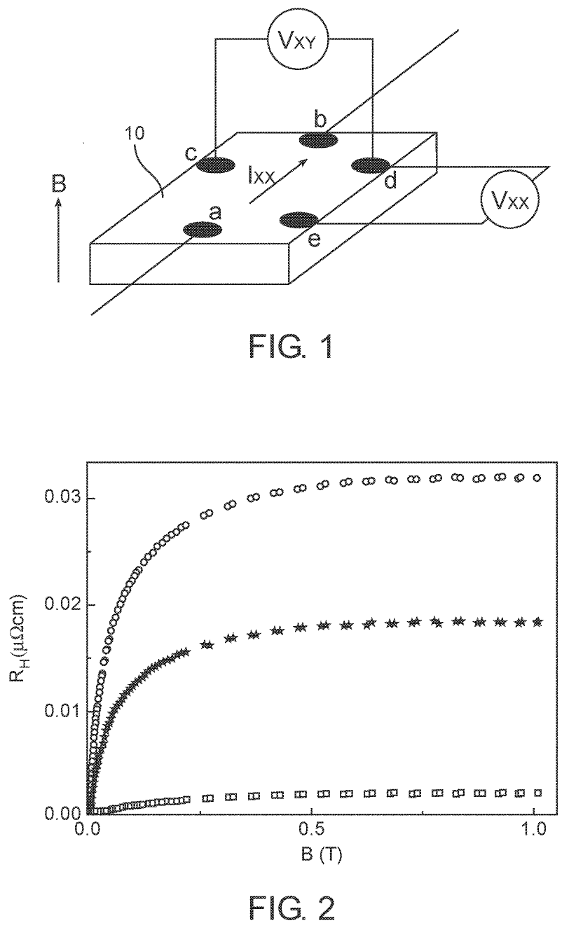

As mentioned, FIG. 1 is a schematic illustration of one exemplary setup according to the invention. Actually, it is one embodiment of the proposed gas sensor. FIG. 1 comprises arrangements for the resistance and the Hall effect measurement in a Hall bar configuration serving for gas detection, with electric current and applied magnetic field orientations, comprising the resistance and the Hall effect measurement circuits in a Hall bar configuration, with electric current and applied magnetic field orientations (one exemplary orientation of the magnetic field is shown by the arrow “B”). The Hall bar (being a conductive layer/film of a gas sensor) is marked 10. Contacts for injecting electric current are marked by small letters a and b. Contacts for measuring a Hall signal are marked with small letters c and d. Voltage V_{XX} is measured between measurement contacts d and e. Resistance of the Hall bar may be extracted from V_{XX} voltage measured along the current flow direction. Hall voltage V_{H} (V_{XY}) may be measured as the Hall signal, across the bar 10 in the direction perpendicular to the current flow.

Dimensions of the Hall bar may be as small as state of the art lithographic technologies can provide (i.e., under 1 μm), but may also reach some centimeters.

FIG. 2 presents a schematic illustration of the extraordinary Hall effect measured as a function of magnetic field at different concentrations of the target gas. Magnetic field is perpendicular to the film plane. In FIGS. 2 to 4, the magnetic field axis is marked “B” (for magnetic induction). Those skilled in the art understand that magnetic field may be interchangeably expressed by B or by H. The Hall signal (EHE resistivity signal) increases with magnitude of the applied field and saturates to a constant value at sufficiently high fields when magnetization of the sensor film reaches saturation in a direction parallel to the direction of applied magnetic field. Magnitude of the saturated EHE signal depends on concentration of the target gas in atmosphere. Therefore, magnitude of the EHE signal measured at any fixed field serves as measure of the target gas concentration. Different concentrations of the target gas are schematically indicated by different geometrical figures (circles, squares, asterisks) used for building the curves. In this example, the sensor film exhibits no hysteresis in the magnetic field perpendicular to its plane. In one embodiment of the invention, the measurement may be performed at sufficiently high magnetic fields when magnetization and the EHE signal are saturated.

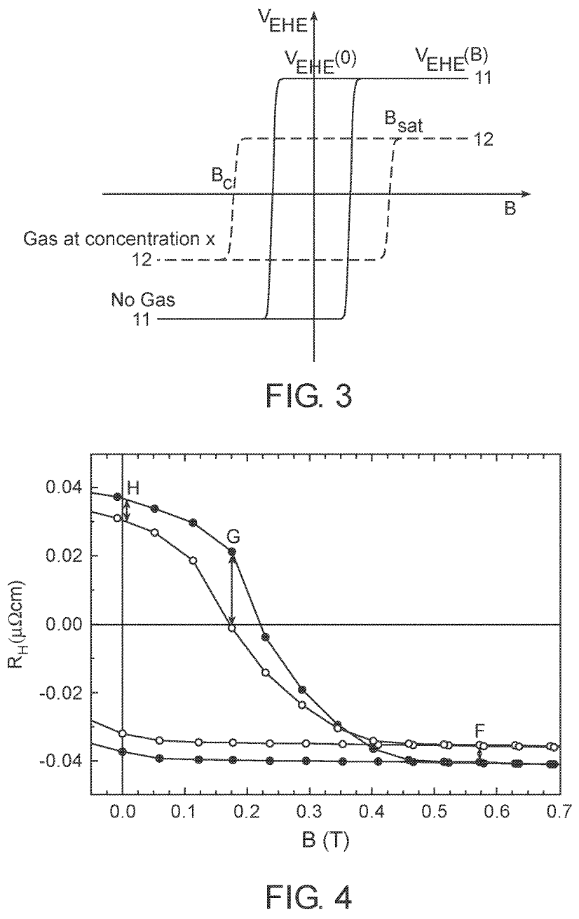

FIG. 3. presents a schematic illustration of another embodiment where the extraordinary Hall effect is measured as a function of magnetic field at different concentrations of the target gas. In this embodiment the sensor film exhibits the out-of-plane magnetic anisotropy and hysteresis in the magnetic field applied perpendicular to its plane.

A hysteresis loop 11 is observed in absence of the target gas. A hysteresis loop 12 is observed when the target gas concentration in air is x.

Several measured parameters that indicate the presence and concentration of the target gas are: magnitude of the EHE signal at high fields V_{EHE}(H); the remnant EHE signal at zero field V_{EHE}(0), which can vary between zero and V_{EHE}(H); the coercive field of the hysteresis loop H_{C} (field at which magnetization and the EHE signal cross zero); and the saturation field H_{SAT}, at which magnetization and the EHE signal reach saturated values. The above parameters can be used for selecting a specific working point for a particular sensor element (See FIG. 4 and the corresponding description).

As mentioned above, the magnetic parameters of the proposed sensor element must be sensitive to the presence of a specific gas. In some embodiments of the present invention, the sensor is configured to record two parameters sensitive to the presence and concentration of gases: longitudinal resistance in addition to Hall effect resistance (in particular the extraordinary Hall resistance). When measuring V_{H}, for improved accuracy, separation between longitudinal resistance and Hall resistance may be achieved by application of a reverse magnetic field reciprocity (RMFR) theorem, which is well known in the art. Longitudinal resistance is an even function of the magnetic field, therefore the longitudinal voltage corresponding to resistance of a sensor follows relation: Vₓₓ(H)=Vₓₓ(−H). The Hall effect voltage is an odd function of magnetic field, meaning: V_{H}(H)=−V_{H}(−H). According to the RMFR theorem for a sample with arbitrary geometrical form with four electric contacts attached at any points a,b,c and d along the perimeter of the sample:

V_{ab,cd}(H)=V_{cd,ab}(−H),

where a,b,c and d are four arbitrary locations in a system, the first pair indicates the current leads and the second the voltage leads. In ferromagnetic materials magnetization replaces the applied magnetic field, giving in our case:

V_{ab,cd}(M)=V_{cd,ab}(−M).

The odd in magnetization EHE term V_{H} can be determined from two measurements of V_{ab,cd} and V_{cd,ab} made at a given field H as:

V_{H}=1/2(V_{ab,cd}−V_{cd,ab})

The longitudinal voltage corresponding to the longitudinal resistance can be determined as: Vₓₓ=1/2(V_{ab,cd}+V_{cd,ab}) where V_{ab,cd} is the voltage measured between points c and d when current is flowing between contacts a and b; and V_{cd,ab} is the voltage measured between points a and b when current is flowing between contacts c and d.

It should be taken into account, that for different materials and gases, the hysteresis loop may take different shapes. With exposure to gas, the magnitude of the saturated EHE signal can increase or decrease and the coercive field (width of the hysteresis loop) can increase or decrease.

FIG. 4 is a zoom of a hysteresis loop of a sensor element being a ferromagnetic sample/film adapted for detecting a target gas. Let the measurements performed in air are marked with black circles, while the measurements in the presence of a target gas are indicated by white circles. The Hall data shown in FIG. 4 is data in positive magnetic fields, with points F, G and H indicating three exemplary possible working points of the sensor device. Point F indicates measurement of the saturated EHE at sufficiently high magnetic field. This high field measurement can be performed using any suitable ferromagnetic film regardless of its magnetic anisotropy. Point G corresponds to measurement of the shift in coercive field of the film and can be done using materials with perpendicular magnetic anisotropy exhibiting hysteresis in perpendicularly applied magnetic field. Point H corresponds to measurement at zero magnetic field and monitors changes in the remnant EHE signal. It is important to note that magnetic saturation occurs in magnetic field below of about 0.5 T, which is in the range of fields that can be produced by modern permanent magnets.

To estimate feasibility of the EHE gas detection, the Inventor studied the EHE response to hydrogen using thin CoPd alloy films. Hydrogen is highly soluble in palladium, and the Inventor considered making palladium the metal of choice in hydrogen sensors. The palladium lattice expands significantly with absorption of hydrogen (0.15% in the α-phase and 3.4% in the β-phase), and resistivity of Pd increases with conversion into palladium hydride. Similar response is also observed in Pd-based alloys. The Inventor's earlier studies of Co-Pd alloys and multilayers revealed a strong sensitivity of the magnitude and polarity of the EHE signal on the relative content of the system, in particular for Co volume concentrations in the range 10%-30% [4]. The Inventor assumed that absorption of hydrogen by palladium will modify the structure and electronic state of the system and thus affect the EHE signal to allow using it for the gas detection.

Polycrystalline CoₓPd₁₋ₓ films with Co atomic concentration x in the range 0≤x≤0.4 were deposited by e-beam co-evaporation from two separate targets on room temperature GaAs substrates. Co and Pd are completely soluble and form an equilibrium fcc solid solution phase at all compositions. Film thickness varied between 5 nm and 20 nm. Several samples were deposited on silicon and glass substrates and demonstrated the response similar to those deposited on GaAs.

The Inventor has found that the CoPd film of interest may be in the form of a Co/Pd bi-layer, a Co/Pd multilayer, a Co-Pd alloy or any combinations thereof.

The Inventor has further found that the CoPd film may have the Co volume concentration in the range of about 3% to 45%, for example when the film is the form of the alloy or the multilayer.

Results of the study, conducted by the Inventor concerning feasibility of the EHE gas detection using a CoPd film, make reasonable also the use of a FePd film and/or a NiPd film for the same purpose.

Feasibility of the approach was demonstrated by detecting low concentration hydrogen using thin CoPd films as the sensor material of the electrically conductive layer. It has been shown that such Hall effect sensitivity of thus optimized samples exceeds 240% per 10⁴ ppm at hydrogen concentrations below 0.5% in the hydrogen/nitrogen atmosphere, which is more than two orders of magnitude higher than the sensitivity of the conductance detection.

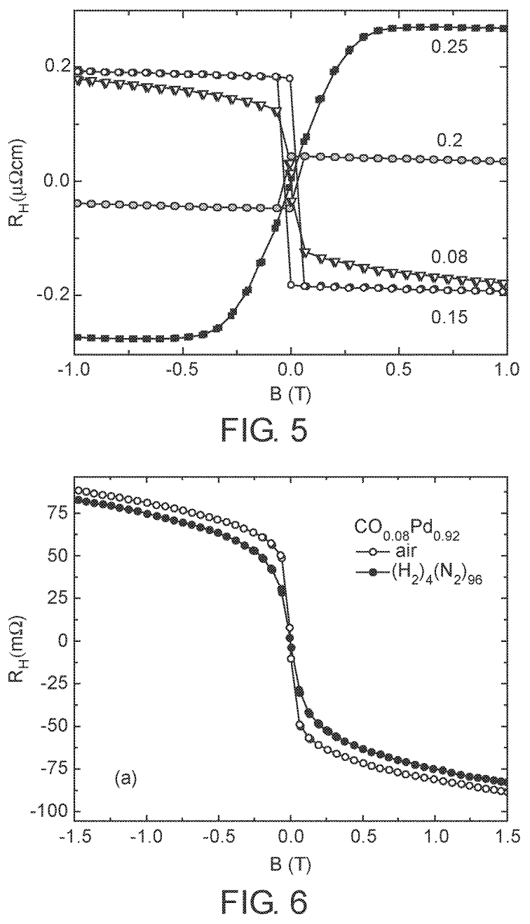

FIG. 5 presents the Hall resistivity ρ_{H}=R_{H}t as a function of magnetic field for four CoₓPd₁₋ₓ samples with x=0.08, 0.15, 0.2 and 0.25 (atomic concentration) and thickness 15nm, 18nm, 15nm and 14nm respectively measured in ambient air at room temperature. The ordinary Hall effect, corresponding to the high field linear slope beyond magnetization saturation, is negligible, and the observed signal is mainly due to the EHE. Polarity of the effect, defined as dρ_{H}/dB, indicates the dominance of the right-hand versus left-hand spin-orbital scattering. The polarity reverses between x=0.15 and x=0.2. Samples richer in Co exhibit a positive polarity, while samples richer in Pd have a negative one. The out-of-plane magnetic anisotropy with a significant hysteresis is developed in samples in a vicinity of the EHE sign reversal point. Development of the perpendicular anisotropy has been attributed to a strained state of thin CoPd films, that are known to have a very large magnetostriction reaching its maximum in the same concentration range.

Replacement of the ambient air by the pure nitrogen or by the pure carbon monoxide CO atmospheres does not affect the EHE loops. However, the response is significant when hydrogen is added.

The four samples shown in FIG. 5 were measured in hydrogen/nitrogen H₂/N₂ mixture with 4% of hydrogen, and the results are presented in FIGS. 6, 7, 8. The magnitude of the saturated EHE signal is reduced by 2% to 15% in all hydrogenated samples. The most pronounced changes are observed in the hysteresis loops of the samples with the out-of-plane anisotropy.

FIG. 6 shows a non-hysteresis example of a CoPd film.

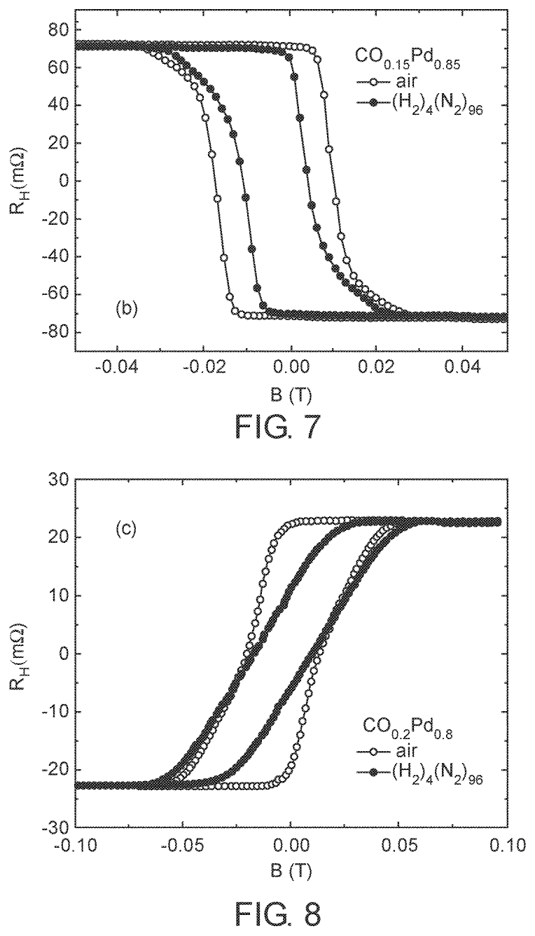

Width of the quadratic hysteresis loop shrinks in the Co_{1.5}Pd_{0.85} sample (FIG. 7).

The Co_{0.2}Pd_{0.8} sample also demonstrates a reduction of the coercive field together with the zero field remanence signal reduced to about a half (FIG. 8). Reduction of the coercive field and the remanence indicate the decreasing perpendicular magnetic anisotropy with hydrogen absorption.

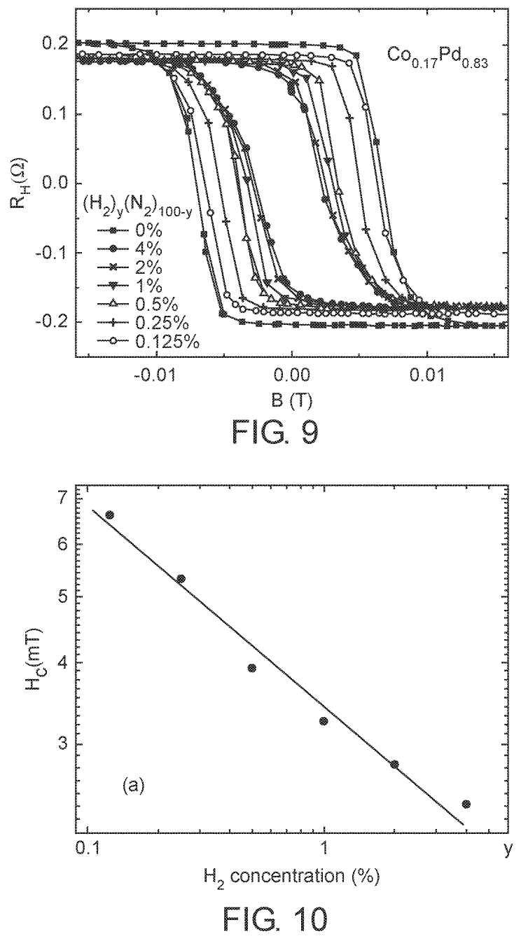

FIG. 9 presents the field dependent hysteresis loops measured in 5 nm thick Co_{0.17}Pd_{0.83} sample in H₂/N₂ atmosphere at different hydrogen concentrations between 0 and 4%. FIG. 9 illustrates absolute changes of the Hall resistance R_{H} for different concentrations of hydrogen. The seven different concentrations are listed in the legend, marked by different geometrical figures and are presented by respective curves on the drawing.

Thinner films seem to be attractive for sensing purposes due to a higher surface to volume ratio, and since the absolute value of the measured signal (Eqs. 1 and 2) increases both by the reducing thickness t and by enhancing the EHE coefficient R_{EHE} boosted by the spin-orbit surface scattering. After the initial measurement in N₂ (99.998%) at atmospheric pressure, the sample chamber was filled with H₂ 4% H₂/N₂ mixture. The following sequence of measurements at reduced hydrogen concentrations was done after pumping the chamber to half of atmospheric pressure and refilling the chamber by nitrogen. After completing the sequence, the sample was re-measured in N₂. The hysteresis loops are fully reproducible when the sequence is repeated. As seen, the saturated magnitude of the signal at high field, the remanence at zero field and the width of the hysteresis loop decrease with increasing hydrogen concentration.

The quantitative data are shown in FIGS. 10-12.

FIG. 10 presents the coercive field H_{c} as a function of hydrogen concentration y. H_{c} can be well presented by the power law dependence on the hydrogen concentration as: H_{c}(y)=H_{c}(0)y^{-y} with H_{c}(0)≈7mT and γ≈0.3, i.e. it varies significantly at low hydrogen concentrations and saturates towards 4%.

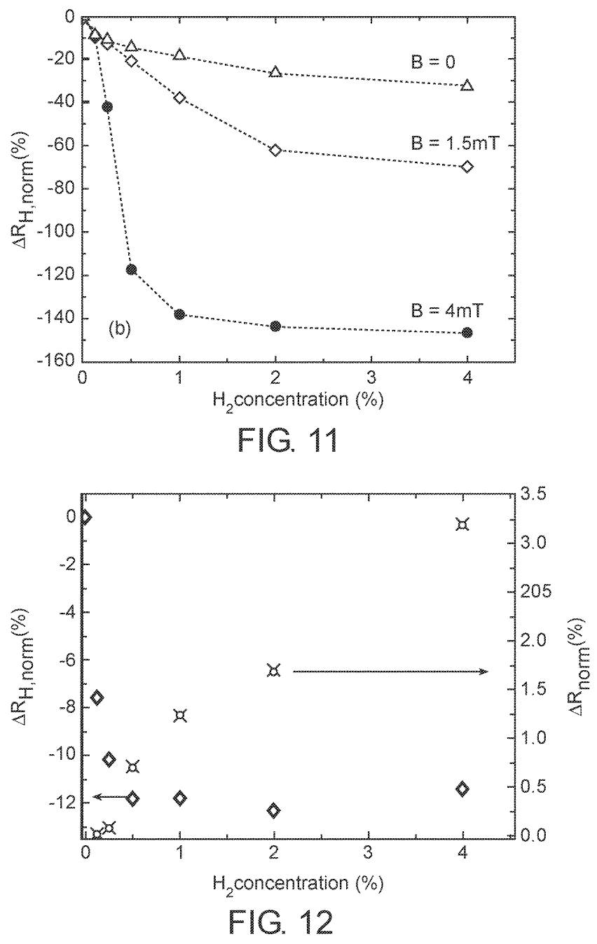

FIG. 11 presents the normalized change of the EHE signal measured at several fixed fields within the hysteresis loop (H=0, 1.5 mT and 4 mT, which represent some three working points). The normalized EHE change is defined as:

$${Δ R}_{H , norm} ( y ) = \frac{{Δ R}_{H} ( y )}{{Δ R}_{H} ( 0 )} = \frac{{Δ R}_{H} ( y ) - {Δ R}_{H} ( 0 )}{{Δ R}_{H} ( 0 )} ,$$

where y is the hydrogen concentration. The signal varies strongly at low H₂ concentrations and saturates by approaching 4%. The rate of the signal variation and the range of the linear response depend on the bias field. At 4 mT bias field the sensitivity (S=dΔR_{H,norm}/dy) exceeds 240%/10⁴ H₂ ppm at hydrogen concentrations below 0.5%. At 1.5 mT the sensitive range extends up to 2% of hydrogen with sensitivity about 30%/10⁴ ppm. Variation of the remnant EHE signal at zero bias field reaches 30% at 4% hydrogen. The response is not linear over a wider concentration range, which should be taken into account in calibration of the future sensors.

It should be kept in mind that FIG. 11 shows relative (normalized) changes of resistance R_{H}, while FIG. 9 showed absolute changes thereof.

FIG. 12 further shows Resistance response to hydrogen, measured simultaneously with the EHE response to hydrogen. FIG. 12 simultaneously presents:

On right vertical axis—Hydrogen concentration dependence of the normalized resistance change ΔRₙₒᵣₘ at zero field (marked as ∘) and under 0.1T bias field (marked as x);

On left vertical axis —Hydrogen concentration dependence of the normalized EHE change ΔR_{H,norm} under 0.1T bias field—left vertical axis, all measured in 5 nm thick Co_{0.17}Pd_{0.83} film.

The Resistance measurements are marked with crosses (x) and circles (∘). The data taken at zero field (marked as ∘) and the data taken in the magnetically saturated state under 0.1T bias field (marked as x) are presented in the form of the normalized resistance change (right vertical axis), defined as:

$${Δ R}_{norm} = \frac{R ( y ) - R ( 0 )}{R ( 0 )} .$$

Both at zero and under 0.1T field resistance increases about linearly with hydrogen concentration up to 4%. The resistance sensitivity to hydrogen concentration, defined as: dΔRₙₒᵣₘ/dy, is about 0.8%/10⁴ ppm. Magnetoresistance of the sample is small, negative and independent on hydrogen absorption. Therefore, the resistance changes caused by hydrogen adsorption don't depend on the bias field.

Measurements of the normalized EHE response ΔR_{H,norm} (left vertical axis) taken in the magnetically saturated state at a fixed field 0.1T, are marked with rhomboids. The EHE and the resistivity responses to hydrogen absorption are independent of each other. The magnetic EHE response is negative, reaches 12% at low hydrogen presence and saturates towards 4% concentration. Resistivity increases in the measured H₂ concentration range with no signs of saturation. Following Eq.2, the EHE signal depends on the EHE coefficient R_{EHE} and magnetization M. R_{EHE} scales with resistivity as: R_{EHE}∝ρ due to the skew scattering mechanism or as R_{EHE}∝ρ², following the intrinsic Berry phase mechanism or the extrinsic mechanism of side jump scattering. Changes of the saturated magnetization and of the field dependent hysteresis loop due to gas absorption are uncorrelated with resistivity, which makes the EHE and resistivity responses independent.

Reduction of the saturated EHE signal with increasing hydrogen concentration is consistent with the generally observed decrease of the total magnetization in hydrogenated Co/Pd systems, the effect attributed to modification of the electronic structure of the material. On the other hand, the effect of hydrogen absorption on the perpendicular anisotropy is ambivalent. Enhancement of the perpendicular magnetic anisotropy was found in hydrogenated Pd/Co/Pd trilayers, associated with improvements of Pd (1,1,1) orientation. The coercive field and the perpendicular magnetic anisotropy of our samples decrease with hydrogen absorption. The Inventor supposes that changes in magnetic anisotropy depend strongly on magnetostriction and strain of the material, similar to the concentration dependence of non-hydrogenated CoPd films (FIG. 5).

To summarize, one can expect that selectivity of solid state gas sensors will be improved by extending the range of independent measurable parameters complementing the conductometric sensing. The extraordinary Hall effect (EHE), sensitive to variations of magnetic properties of ferromagnetic materials, can serve both as an independent and as a complementary magnetotransport parameter. Especial advantage of the proposed technique for EHE-based gas sensing was demonstrated here by its capability of detecting low concentration hydrogen using thin CoPd films. Sensitivity of the EHE response in the optimized samples exceeds 240% per 10⁴ ppm at hydrogen concentrations below 0.5% in the hydrogen/nitrogen atmosphere, which is more than two orders of magnitude higher than the sensitivity of the conductance detection.

The combined use of the EHE and conductance detection is justified at higher concentrations of hydrogen, for example between 1% and 100% where the EHE detection becomes less effective.

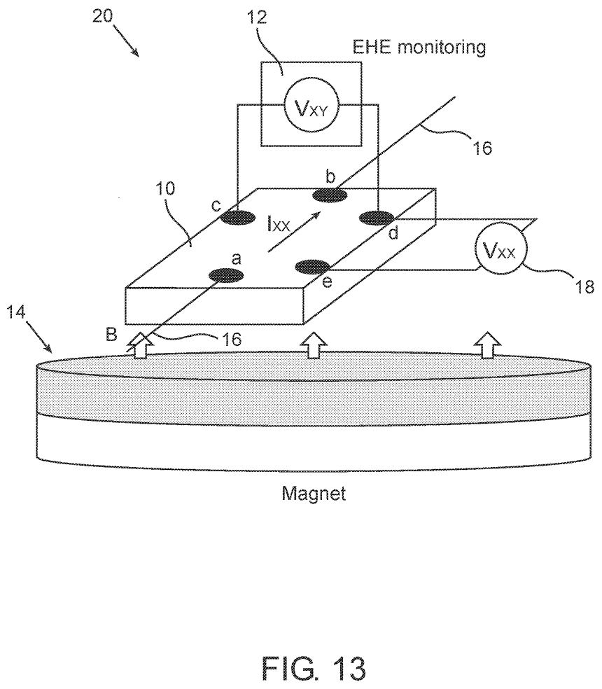

FIG. 13 is a schematic illustration of one embodiment of the proposed gas sensor device 20 incorporating the sensor element comprising a film in the form of a Hall bar 10, at least a first measuring circuit 12 for monitoring an EHE signal and a magnetic field generator 14—for example in the form of a permanent magnet located in close proximity to the film 10. Contacts 16 are intended for connecting to a power source (not shown), for injecting electric current into the film 10.

An optional processing/analytical circuit (not shown in this embodiment) may collect and process information received from at least the first measuring circuit 12. Direction of the electric current injected in the Hall bar 10 is marked with I_{XX}.

An optional second measuring circuit 18 is shown in FIG. 13 for measuring conductivity of the sensor element 10. More specifically, the optional circuit may be adapted for measuring a longitudinal voltage V_{XX} in the direction of I_{XX}, while the main measuring circuit may be adapted for measuring Hall voltage V_{H} in the direction perpendicular to the I_{XX} direction. The Hall voltage is marked as V_{XY} in FIG. 13.

The first (main) measuring circuit 12 which allows monitoring the EHE signal may actually serve as a detector of the target gas. The main measuring circuit 12 may incorporate or may be connected to a displaying device (not shown).

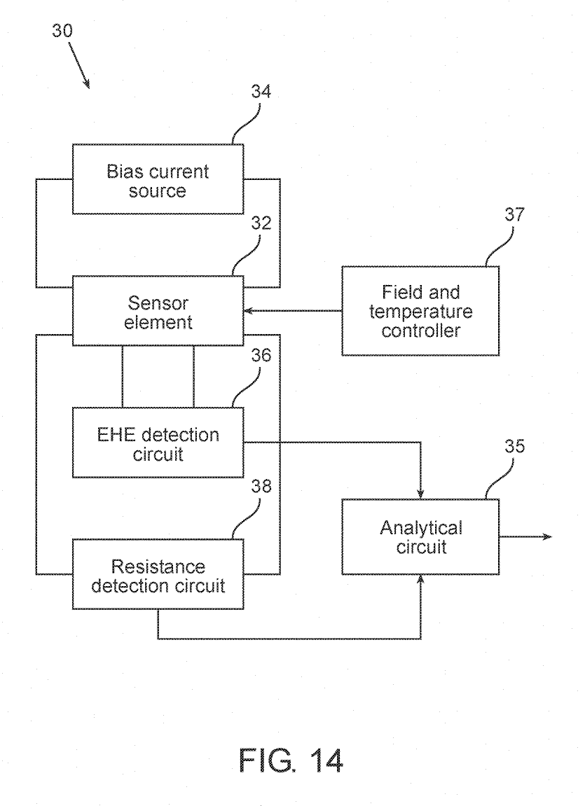

FIG. 14 illustrates a schematic block diagram of another embodiment 30 of the proposed gas sensor device. The embodiment comprises a sensor 32 (e.g., the gas sensor element 10 shown in FIGS. 1 and 13), a bias current source 34, an EHE detection circuit 36 (e.g., the main measuring circuit 12), a Resistance detection circuit 38 (e.g., an optional measurement circuit 18), wherein both the EHE detection circuit and the Resistance detection circuit are connected to an Analytical circuit 35, for processing the information obtained from at least the circuit 36 (and optionally also from circuit 38). The sensor device 30 schematically shown in FIG. 14 also comprises a field and temperature controller 37, which controls a) magnetic field applied to the sensor element 32 for example from a generator of external magnetic field (for example, like 14; not shown in this drawing), b) temperature of the sensor element.

It should be noted, that usually a target gas reacts with a thin film at a specific temperature range. In another example, for “cleaning” the sensor from a specific gas, it should be heated up to a specific temperature. To enable those options, controller 37 may be configured both to provide and monitor predetermined temperature ranges at the sensor 32. The controller 37 may also incorporate the magnetic field generator. The analytical/processing circuit 35 is designed to process the obtained information and to produce a report on the gas detection result (about presence and optionally, concentration of a target gas).

It should be appreciated that though the invention was described with reference to a number of specific embodiments, other embodiments of the sensor element, of the sensor, other versions of the method may be proposed and should be considered part of the invention whenever defined by the claims which follow after the list of prior art references.

### REFERENCES

1. C. S. Chang, M. Kostylev, and E. Ivanov, Metallic spintronic thin film as a hydrogen sensor, Appl. Phys. Lett. 102, 142405 (2013).

2. A. K. Schmid, A. Mascaraque, B. Santos, J. de la Figuera, Gas sensor, US Patent Application US 2012/0131988 A1 (2012)

3. A. Gerber, Magnetic Thin Film Sensor Based on the Extraordinary Hall Effect, U.S. Pat. No. 6,794,862 B2 (2004), U.S. Pat. No. 7,110,216 B2 (2006), U.S. Pat. No. 7,463,447 B2 (2008).

4. G. Winer, A. Segal, M. Karpovski, V. Shelukhin, and A. Gerber, J. Appl. Phys. 118, 173901 (2015).

1. A method for determining presence and/or concentration of a target gas by monitoring changes of Extraordinary Hall Effect (EHE) exhibited by an electrically conductive layer of a sensor element in presence of the target gas.

2. The method according to claim 1, comprising magnetizing said layer, providing passage of electric current through said layer, wherein said monitoring of EHE changes includes monitoring of the EHE exhibited in a direction perpendicular to direction of said electric current, without and supposedly with said target gas.

3. The method according to claim 1, wherein the monitoring of changes in the EHE is performed by measuring a Hall effect signal in the form of Hall voltage or Hall current.

4. The method according to claim 1, comprising:
providing said sensor element comprising a conductive layer capable of exhibiting EHE,
magnetizing said sensor element,
providing passage of electric current through said conductive layer,
monitoring the changes in EHE based on measuring a first electric signal being a Hall effect signal exhibited in a direction perpendicular to said electric current's direction, and
determining the presence and concentration of the target gas by processing the measured first electric signal.

5. (canceled)

6. The method according to claim 4, further comprising monitoring changes of conductivity of the sensor element by measuring a second electric signal in said electric current's direction, and determining the presence and/or concentration of the target gas by processing the first and the second electric signals.

7. The method according to claim 1, performed under a predetermined magnetic field or a sequence of predetermined magnetic fields.

8. The method according to claim 1, comprising:
providing at least one additional electrically conductive unit made of a different material or composition of materials than the basic electrically conductive layer, said additional unit being capable of exhibiting Extraordinary Hall Effect (EHE) changeable in presence of said target gas or an additional target gas;
performing the monitoring step separately for the basic electrically conductive layer and for the additional unit,
the method thereby allows either detecting said target gas with higher sensitivity, or detecting two or more target gases.

9. The method according to claim 1, wherein the electrically conductive layer comprises a material selected from CoPd, FePd, NiPd or combinations thereof for detecting presence and/or concentration of hydrogen.

10. The method according to claim 9, for detecting low concentrations of hydrogen from 0% to 4%.

11. A gas sensor element comprising an electrically conductive layer capable of exhibiting Extraordinary Hall Effect (EHE) changeable in response to presence and/or concentration of a target gas.

12. The gas sensor element according to claim 11, wherein said electrically conductive layer is capable of exhibiting said changeable EHE upon being magnetized and exposed to electric current through said conductive layer.

13. (canceled)

14. The gas sensor element according to claim 11, wherein the electrically conducting layer of the sensor element comprises one or more of the following materials: ferromagnetic material, like ferromagnetic metal or a ferromagnetic semiconductor; paramagnetic material, bilayer or multilayer, alloy or mixture of magnetic and/or non-magnetic materials.

15-16. (canceled)

17. The gas sensor element according to claim 11, comprising one or more additional electrically conductive units, each exhibiting EHE being changeable in response to a specific target gas.

18. The gas sensor element according to claim 17, wherein said one or more additional electrically conductive units are responsive to said target gas.

19. The gas sensor element according to claim 17, wherein said conductive layer and said one or more additional conductive units are made of the same material/composition of materials and are arranged in a stack of conductive layers divided by protective layers and having a common input contact and a common output contact for injecting electric current through said conductive layers.

20. The gas sensor element according to claim 17, wherein said electrically conductive layer and said one or more additional electrically conductive units made of different materials or compositions.

21. The gas sensor element according to claim 11, with the electrically conductive layer comprising a material selected from CoPd, FePd, NiPd or combinations thereof for detecting presence and/or concentration of hydrogen.

22-26. (canceled)

27. The gas sensor element according to claim 21, for detecting low concentration hydrogen, from 0% to 4%.

28. A set of gas sensor elements, comprising two or more of the gas sensor elements each designed according to claim 11, for respectively detecting presence and/or concentration of two or more different target gases.

29. A device comprising a gas sensor element having an electrically conductive layer capable of exhibiting Extraordinary Hall Effect (EHE) changeable in the presence of a target gas; the device being adapted to monitor changes of the EHE for determining presence and/or concentration of the target gas.

30. The device according to claim 29, comprising:
a magnetic field generator for magnetizing the gas sensor element,
contacts for connecting said conductive layer to an electric power source to pass electric current through the layer,
a measuring unit comprising at least a first circuit for monitoring the EHE changes by measuring a first electric signal being a Hall effect signal in a direction perpendicular to the electric current's direction.

31. (canceled)

32. The device according to claim 29, further comprising a processing unit configured to determine the presence and/or concentration of the target gas based on changes of the first electric signal.

33. The device according to claim 30, further comprising a second circuit for monitoring conductivity of the conductive layer by measuring a second electric signal indicative of resistance along the conductive layer, and the processing unit configured to define the presence and concentration of the target gas based on changes of the first electric signal and changes of the second electric signal.

34. The device according to claim 33, comprising:
the first circuit for monitoring Hall voltage V_{H} in a direction perpendicular to the current direction, and a second circuit for monitoring electric voltage Vₓₓ in the direction of electric current;
the processing unit for processing the monitored values of electric voltage Vₓₓ and Hall voltage V_{H} and for judging about the presence and concentration of said at least one target gas, based on the processing results.

35. The device according to claim 30, wherein the magnetic field generator is configured to produce a constant magnetic field applied to the electrically conductive layer.

36. The device according to claim 30, wherein the magnetic field generator is configured to produce continuously variable magnetic field or a sequence of magnetic field pulses, so as to alternate the magnetic field in polarity and in magnitude along the sequence.

37. The device according to claim 32, wherein the processing unit is configured to apply a reverse magnetic field reciprocity (RMFR) theorem to the measurements of V_{H} collected from said layer so as to separate, from the measured vector of V_{H}, a component of Vxx indicating the first longitudinal resistance, and a component of the second Hall voltage V_{H}.

38. The device according to claim 30, designed to accommodate multiple sensor elements sensitive to multiple different target gases; the measuring unit of the device being accordingly designed to ensure monitoring of changes of EHE respectively exhibited by said multiple sensor elements.

39. The device according to claim 29, comprising the gas sensor element with the electrically conductive layer comprising a material selected from CoPd, FePd, NiPd or combinations thereof, the device being adapted for detecting presence and/or concentration of hydrogen.

40. A software product comprising computer implementable instructions and/or data for carrying out the method according to claim 1, being stored on an appropriate non-transitory computer readable storage medium so that the software is capable of enabling operations of said method when used in a computerized system.
